(19) United States
(12) Patent Application Publication
(10) Pub. No.: US 2010/0144596 A1
(43) Pub. Date: Jun. 10, 2010

(54) An Apo-Ferredoxin Preparation, A Process For Producing Same And Uses Thereof

(75) Inventor: Rachel Nechustai, Motza Elite (IL)

Correspondence Address: MARTIN D. MOYNIHAN d/b/a PRTSI, INC., P.O. BOX 16446, ARLINGTON, VA, 22215, US

(73) Assignee: Yissum Research Development Company of the Hebrew University of Jeruslem Ltd., Jerusalem (IL)

(21) Appl. No.: 12/448,358

(22) PCT Filed: Dec. 18, 2007
(86) PCT No.: PCT/IL2007/001559

§ 371 Date: Dec. 23, 2009

Related U.S. Application Data
(60) Provisional application No. 60/875,537, filed on Dec. 19, 2006.

(51) Int. Cl. A61K 38/16; C07K 14/00; C07K 1/18; G01N 33/20; A61P 7/00; C05F 11/00

(52) U.S. Cl. 514/6; 530/400; 530/416; 436/84; 71/27

(57) ABSTRACT

A process for obtaining a pure preparation of an apo- form of an [Fe₂S₂] ferredoxin is provided. The process comprising, applying a solution comprising a holo- form of the [Fe₂S₂] ferredoxin on an anion exchange column; and eluting the [Fe₂S₂] ferredoxin from the anion exchange column with a solution comprising a salt, thereby obtaining the pure preparation of the apo- form of the [Fe₂S₂] ferredoxin. Also provided are pure preparations of apo-ferredoxin as well as methods of using same.

### FIELD AND BACKGROUND OF THE INVENTION

The present invention, in some embodiments thereof, relates to a preparation of an iron-sulfur protein and, more particularly, but not exclusively, to an iron-sulfur protein useful as a chelator.

Iron-sulfur proteins are proteins which naturally comprise iron-sulfur clusters. Iron-sulfur clusters comprise iron ions bound to sulfide ions. Typically, the iron-sulfur cluster is coordinated by the sulfur atoms of cysteine residues, or by a combination of histidine residue nitrogen atoms and cysteine residue sulfur atoms. The iron ions may be in a Fe³⁺ or Fe²⁺ state, and hence iron-sulfur clusters may undergo redox reactions.

Several types of iron-sulfur clusters are known to occur in proteins. The [Fe₂S₂] cluster consists of two iron ions bridged by two sulfide ions, typically coordinated by four cysteine residues or by two cysteine residues and two histidine residues. The four sulfur atoms are organized in an approximately tetrahedral arrangement around each iron atom. [Fe₂S₂] clusters in proteins typically comprise either two Fe³⁺ ions or one Fe²⁺ and one Fe³⁺ ion.

The [Fe₄S₄] cluster consists of four iron ions and four sulfide ions in a cubic arrangement, and is typically coordinated by four cysteine residues. Typically, 1, 2 or 3 of the iron ions are Fe³⁺, and the rest are Fe²⁺.

The [Fe₃S₄] cluster consists of three iron ions and four sulfide ions in a cubic arrangement, typically coordinated by three cysteine residues.

Examples of the structures of [Fe₂S₂], [Fe₃S₄] and [Fe₄S₄] clusters are shown in FIG. 1.

Ferredoxins (Fd) are soluble iron-sulfur proteins, found in bacteria, plants, and mammalian cells, which are involved in numerous electron transfer reactions. Ferredoxins are commonly characterized and classified by the type of iron-sulfur cluster which they comprise. Ferredoxins from plants, algae, and photosynthetic bacteria, denoted as plant-type ferredoxins, have a single [Fe₂S₂] cluster. The three-dimensional structure in the vicinity of the iron-sulfur cluster is highly conserved in many plant-type ferredoxins [Knaff and Hirasawa (1991); Holden et al. (1994)]. The oxidation-reduction midpoint potential (Eₘ) values of these ferredoxins are typically around −0.42 eV [Cammack et al. (1977)], making the reduced form of these ferredoxins one of the strongest soluble reducing agents known in nature. The ferredoxin serves as the electron donor in several essential reactions such as NADP⁺ reduction, carbon assimilation, nitrite reduction for nitrogen assimilation, sulfite reduction, glutamate synthesis and thioredoxin reduction for metabolism regulation [Knaff and Hirasawa]. The reduction of plant-type ferredoxin is directly accomplished by the photosystem I reaction center during oxygenic photosynthesis [Setif et al. (2002)].

Ferredoxin isolated from the thermophilic cyanobacterium Mastigocladus laminosus, has been shown to display thermostable properties, with maximal activity at 65° C. [Hase et al. (1978)].

Nishio and Nakai (2000) describe the preparation of the apo- form of Synechocystis ferredoxin by boiling purified holoferredoxin in the presence of 100 nM EDTA and 500 mM dithiothreitol to trap iron atoms liberated from the holoproteins and ensure that the side chains of the four cysteines previously participating in [Fe₂S₂] cluster ligation are reduced to free sulfhydryl groups, followed by purification by gel filtration column chromatography.

Li et al. (1990) describe the preparation of the apo- form of spinach ferredoxin by precipitating holoferredoxin with 10% trichloroacetic acid to remove the iron-sulfur acid, followed by washing with 1% trichloroacetic acid and resuspending the protein in Tris/HCl buffer, pH 7.3.

Busch et al. (2000) describe the preparation of the apo- form of Desulfovibrius africanus ferredoxin III, which comprises a [Fe₃S₄] cluster and a [Fe₄S₄] in the holo- form, by expressing a histidine-tagged form of the protein in E. coli. Busch et al. (2000) further teach that DE 52 anion exchange chromatography leads to cluster loss in the reconstituted D. africanus holoferredoxin III.

Aono et al. (1989) describe the preparation of the apo- form of a thermostable ferredoxin from Pyrococcus furiosus, which comprises 2 [Fe₄S₄] clusters in the holo- form, by incubating the holoferredoxin in 8% trichloroacetic acid.

Chelators are used for a variety of purposes, such as chemical analysis, water purification and chelation therapy (i.e. the use of a chelator for detoxification of metals in the body).

Small organic compounds are commonly used as chelators. For example, ethylenediaminetetraacetic acid (EDTA) is widely used as a chelator for a variety of purposes, and dimercaptosuccinic acid is commonly used for chelation therapy.

### SUMMARY OF THE INVENTION

Some embodiments of the present invention pertain to novel preparations of an apo- form of a ferrodoxin, as well as novel methods of producing and utilizing such preparations.

According to an aspect of some embodiments of the present invention there is provided a pure preparation of an apo- form of a ferredoxin, the preparation exhibiting thermal stability at about 70° C.

According to an aspect of some embodiments of the present invention there is provided a process for obtaining a pure preparation of an apo- form of an [Fe₂S₂] ferredoxin, the process comprising:

(a) applying a solution comprising a holo- form of the [Fe₂S₂] ferredoxin on an anion exchange column; and

(b) eluting the [Fe₂S₂] ferredoxin from the anion exchange column with a solution comprising a salt, thereby obtaining the pure preparation of the apo- form of the [Fe₂S₂] ferredoxin. According to an aspect of some embodiments of the present invention there is provided a pure preparation of an apo- form of an [Fe₂S₂] ferredoxin produced according to the above process.

According to an aspect of some embodiments of the present invention there is provided a method of chelating iron ions from a solution, the method comprising contacting the solution with a preparation described hereinabove, thereby chelating the iron ions from the solution.

According to some embodiments of the invention, the solution is an aqueous solution.

According to an aspect of some embodiments of the present invention there is provided a method of fertilizing a soil, the method comprising:

(a) chelating an iron ion with a preparation described hereinabove; and

(b) depositing the iron ion chelated by the apo-from of the ferredoxin on the soil;

thereby fertilizing the soil.

According to an aspect of some embodiments of the present invention there is provided a method of chelating free iron ions in a subject in need thereof, the method comprising administering an effective amount of a preparation described hereinabove to the subject, thereby chelating free iron ions in the subject.

According to an aspect of some embodiments of the present invention there is provided a method of detecting an iron ion in a sample, the method comprising:

contacting the sample with a preparation described hereinabove; and

detecting the presence of an iron ion chelated by the apo- form of the ferredoxin,

thereby detecting an iron ion in the sample.

According to some embodiments of the invention, the apo- form of the ferredoxin is attached to a label sensitive to the presence of the iron ion chelated by the apo- form of the ferredoxin.

According to some embodiments of the invention, the apo- form of ferredoxin comprises a native apoferredoxin.

According to some embodiments of the invention, the ferredoxin comprises an [Fe₂S₂] ferredoxin.

According to some embodiments of the invention, the [Fe₂S₂] ferredoxin is derived from Mastigocladus laminosus.

According to some embodiments of the invention, the salt comprises a chloride salt.

According to some embodiments of the invention, the chloride salt comprises NaCl.

According to some embodiments of the invention, the solution comprising a salt comprises a concentration of chloride salt that ranges from about 0.25 to about 1 M.

According to some embodiments of the invention, the solution comprising a salt comprises a concentration of chloride salt that ranges from about 0.25 to about 0.4 M.

According to some embodiments of the invention, the process further comprises collecting a fraction which comprises the apo- form of the [Fe₂S₂] ferredoxin following the eluting.

According to some embodiments of the invention, the apo- form of the [Fe₂S₂] ferredoxin exhibits thermal stability at about 70° C.

According to some embodiments of the invention, the hole- form of the [Fe₂S₂] ferredoxin is a recombinant protein expressed in bacteria.

According to some embodiments of the invention, the ferredoxin is attached to a solid substrate.

According to some embodiments of the invention, the subject has an iron overload disorder.

According to some embodiments of the invention, the iron overload disorder is selected from the group consisting of siderosis, hemochromatosis, aceruloplasminemia, atransferrinemia, transfusional iron overload, iron overload associated with chronic liver disease, porphyria cutanea tarda, African iron overload and iron poisoning.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a drawing presenting the structures of an [Fe₂S₂] cluster, an [Fe₃S₄] cluster and an [Fe₄S₄] cluster;

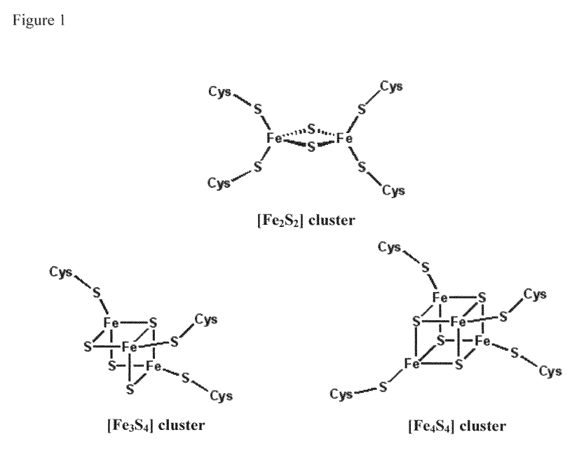

FIG. 2 is a graph presenting an elution profile of ferredoxin from an anion exchange column; the magenta line represents the absorption profile at a wavelength of 260 nm; the blue line represents the absorption profile at a wavelength of 280 rim; the red line represents the absorption profile at a wavelength of 420 nm; and the black line represents the salt concentration;

FIGS. 3a and 3b are graphs presenting an elution profile of the proteins of Peak 1 (FIG. 3a) and Peak 2 (FIG. 3b) respectively; the magenta line represents the absorption profile at a wavelength of 260 nm; the blue line represents the absorption profile at a wavelength of 280 nm; the red line represents the absorption profile at a wavelength of 420 nm;

FIG. 4 is a photograph presenting an SDS-PAGE analysis of the proteins of Peak 1 (lane 1) and Peak 2 (lane 2), as well as samples of 5 μg and 2 μg, respectively, of M. laminosus ferredoxin (lanes C); molecular weight markers (lane MW) indicate that all the proteins have a molecular weight of approximately 14 kDa; and

FIGS. 5a and 5b are drawings presenting the structures of an [Fe₂S₂] cluster site in a holoprotein (FIG. 5a) and apoprotein (FIG. 5b) respectively.

### DESCRIPTION OF SPECIFIC EMBODIMENTS OF THE INVENTION

The present invention, in some embodiments thereof, relates to a preparation of an iron-sulfur protein and, more particularly, but not exclusively, to an iron-sulfur protein useful as a chelator.

As used herein, the phrase “iron-sulfur protein” relates to a protein which comprises an iron-sulfur cluster in a holo- form of that protein or an apo- form of that protein.

As used herein, the terms “apo- form” and “apoprotein”, as well as any other use of the prefix “apo-”, describe a form of a protein which lacks an iron-sulfur cluster that is present in another form (i.e. the holo- form) of that protein. Thus, “apoferredoxin” describes ferredoxin in a form which lacks an iron-sulfur cluster.

As used herein, the terms “holo- form” and “holoprotein”, as well as any other use of the prefix “holo-”, describe a form of a protein comprising an iron-sulfur cluster as part of the protein structure. Thus, “holoferredoxin” describes ferredoxin in a form which comprises an iron-sulfur cluster.

The inventor of the present invention has surprisingly discovered, as described in detail in the Examples section hereinbelow, that the iron-sulfur cluster of a ferredoxin, including thermally stable ferredoxin, may be removed readily from the protein by anion exchange chromatography, resulting in an apoferredoxin. The inventor has further discovered that the apoferredoxin is an effective chelator.

According to embodiments of the present invention, it is thus possible to obtain apoferredoxin without subjecting ferredoxin to denaturing conditions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

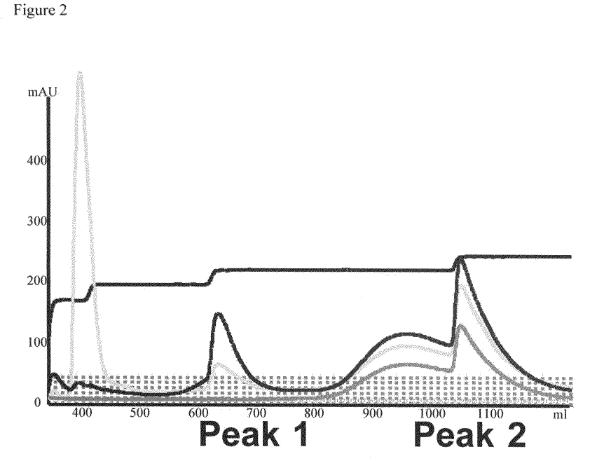

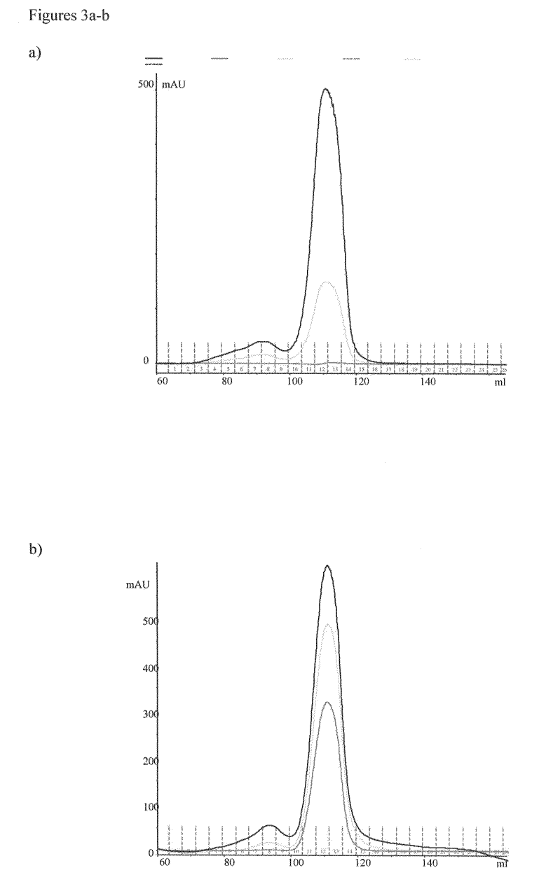

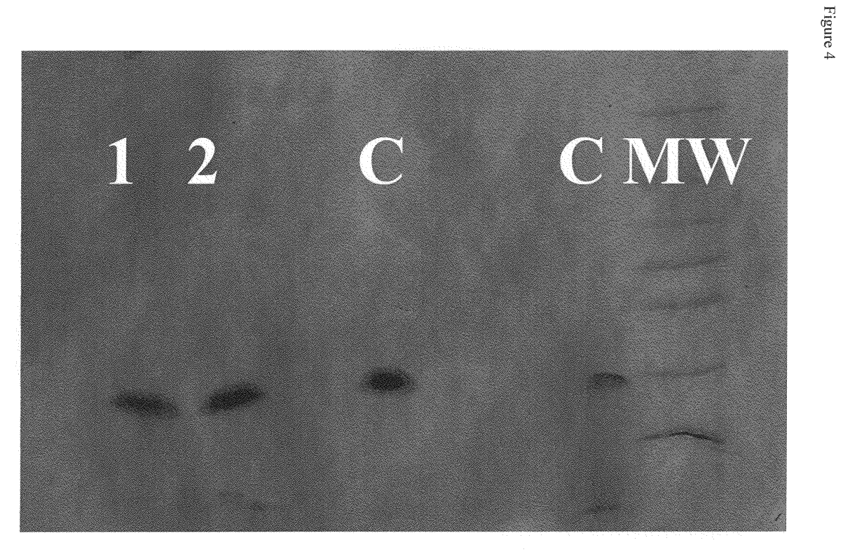

As shown in FIGS. 2 and 3, elution of ferredoxin from an anion exchange column resulted in two protein peaks, one of which comprised an iron-sulfur cluster and one of which lacked an iron-sulfur cluster. As shown in FIG. 4, both proteins were shown by gel electrophoresis to be ferredoxin, indicating that one protein peak represented the apo- form and one represented the holo- form. The elution process thus resulted in a pure preparation of an apoferredoxin and a pure preparation of a holoferredoxin.

According to one aspect of the embodiments of the present invention, there is provided a pure preparation of an apo- form of a ferredoxin, the preparation exhibiting thermal stability at about 70° C.

As used herein the term “ferredoxin” relates to an iron-sulfur protein which mediates electron transfer reactions. Exemplary ferredoxins include, but are not limited to, [Fe₂S₂] ferredoxins (e.g. InterPro No. IPR001041), [Fe₃S₄] ferredoxins (e.g. InterPro No. IPR001080) and [Fe₄S₄] ferredoxins (e.g. InterPro No. IPR001450).

As used herein, the phrase “[Fe₂S₂] ferredoxin” describes any ferredoxin which comprises at least one [Fe₂S₂] iron sulfur cluster in the holo- form of that ferredoxin.

As used herein, the phrase “pure preparation” describes a solid or liquid preparation of a particular protein in which at least about 50%, about 75%, about 90%, about 95%, and even about 99% of the total protein in the preparation is the abovementioned particular protein. The abovementioned “particular protein” may refer to a particular form (e.g. an apo- form) of a particular protein, such that at least about 50%, about 75%, about 90%, about 95%, and even about 99% of the total protein in the preparation is the abovementioned particular form of the particular protein.

For a pure preparation of a protein to exhibit thermal stability at about 70° C., it is meant that the protein of the preparation is not denatured, and exhibits a biological activity (e.g., iron chelation) when the preparation is heated to a temperature of about 70° C. Preferably, the preparation exhibits stability at all temperatures ranging from about 25° C. to about 70° C. or alternatively 40-70° C.

As used herein, the term “about”, when used in the context of a temperature, means ±10° C., preferably ±5° C., and optionally ±2° C.

Optionally, the preparation which exhibits thermal stability at about 70° C. also exhibits thermal stability at about 80° C., at about 90° C., and even at about 100° C.

Because proteins typically lose their structure and biological activity at high temperatures, the thermal stability of the abovementioned preparations allows the preparations to be useful at temperatures at which typical apoferredoxin preparations would have little use.

Preparations of apoferredoxin (as well as other iron-sulfur proteins) are typically prepared by denaturing the holoprotein, as the denatured protein does not bind iron-sulfur clusters as strongly as the native protein. The denatured apoprotein must then be converted into the desired native apoprotein. However, denaturation may not be completely reversible, resulting in reduced yields of native apoprotein and/or alterations in the apoprotein structure and activity.

According to embodiments of the present invention, denatured protein may be avoided.

Thus, according to a preferred embodiment of the present invention, the abovementioned apo- form of a ferredoxin comprises a native apoferredoxin. Preferably, at least about 90% of the apoferredoxin is native, more preferably at least about 95%, and most preferably, at least about 99%.

As used herein, the term “native” describes a protein in a non-denatured form. One of ordinary skill in the art will be familiar with many methods of determining a percentage of native protein (e.g. spectroscopic methods and iron chelation assays). In addition, the apoferredoxin may be assumed to be native if the apoferredoxin was never exposed to conditions known to cause protein denaturation, such as temperatures in excess of the known range of thermal stability of the protein, extreme pH (e.g. below about 5 or above about 10), a non-aqueous solution, high concentrations (e.g. > about 1 M) of urea and/or salt, particularly heavy metal and/or guanidinium salts.

According to a preferred embodiment of the present invention, the abovementioned ferredoxin comprises an [Fe₂S₂] ferredoxin. Preferably, the [Fe₂S₂] ferredoxin is derived from a thermophilic organism such as Mastigocladus spp. (e.g. Mastigocladus laminosus), Thermus spp., Synechococcus spp., Aquifex spp., Hydrogenobacter spp., Thermofilum spp., Hyperthermus spp., Thermotoga spp., Thermosipho spp. and Staphylothermus spp.

The above-described preparation can be mass-produced according to the following novel method of production although it is appreciated that it is applicable to any [Fe₂S₂] ferredoxin as defined hereinabove.

Thus, according to another aspect of the embodiments of the present invention, there is provided a process for obtaining a pure preparation of an apo- form of an [Fe₂S₂] ferredoxin, the process comprising applying a solution comprising a holo- form of the [Fe₂S₂] ferredoxin on an anion exchange column, and eluting the [Fe₂S₂] ferredoxin from the anion exchange column with a solution comprising a salt. Optionally, the solution used for eluting is a chloride salt (e.g. NaCl). A chloride salt is optionally present in the solution at a concentration that ranges from about 0.25 M to about 1 M, and optionally from about 0.25 M to about 0.4 M.

As used herein, the phrase “anion exchange column” encompasses any substance, compound and/or article of manufacturing used in the art to perform anion exchange chromatography.

According to an optional embodiment of the present invention, the process further comprises collecting an eluted fraction which comprises the apo- form of the [Fe₂S₂] ferredoxin. The presence of the apoferredoxin may be determined according to any method known in the art. For example, the ferredoxin may be identified by electrophoresis, while the apo- form may be identified by spectroscopy (e.g. by lack of absorption characteristic of the iron-sulfur cluster).

According to an optional embodiment of the present invention, the process is for obtaining a pure preparation of an apo- form of an [Fe₂S₂] ferredoxin which exhibits stability at about 70° C.

The solution comprising a holo- form of the [Fe₂S₂] ferredoxin may be obtained according to any method known in the art. Optionally, the holo- form is a recombinant protein obtained by being expressed in bacteria.

According to another aspect of the embodiments of the present invention, there is provided a pure preparation of an apo- form of an [Fe₂S₂] ferredoxin produced according to the process described hereinabove.

The inventor of the present invention has surprisingly discovered that the preparations described hereinabove may be used to effectively chelate metal ions (e.g. iron ions). The preparations comprise a protein as a chelator, and are thus to environmentally friendly and biodegradable.

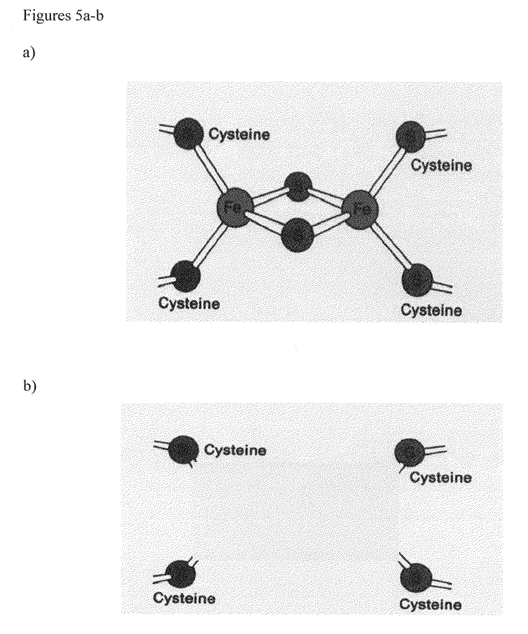

Without being bound by any particular theory, it is believed that the cysteine residues at the site of the iron-sulfur cluster are free in the apoprotein to bind the metal ion, which can fit into the space occupied by the iron-sulfur cluster in the holoprotein. At least some of the cysteine residues may be in an anionic form, thereby facilitating binding to the positively charged metal ion. The hypothesized structure of the chelation site in the apoprotein, in comparison to the structure of the iron-sulfur cluster in the holoprotein, is depicted in FIG. 5.

As used herein, the phrase “iron ions” encompasses all iron ions, e.g. ferric and ferrous ions.

Thus, in another aspect of the embodiments of the present invention, there is provided a method of chelating iron ions from a solution, the method comprising contacting the solution with any of the preparations described hereinabove. Optionally, the solution is an aqueous solution. An exemplary use of chelating iron ions is water purification.

The use of the preparations of the present invention as protein chelators is especially advantageous since they exhibit high thermal stability and as such can endure extreme environmental conditions as well as being non-toxic.

Thus, according to an optional embodiment of the present invention, soil may be fertilized by chelating an iron ion with any of the preparations described hereinabove, and depositing the iron ion chelated by the apoferredoxin on the soil. The iron ion may be slowly released from the ferredoxin, for example, by biodegradation of the protein. Degradation of the protein will not result in harmful byproducts, as is often the case with synthetic compounds, but will result in amino acids, which may further contribute to fertilization of the soil. Release of the iron ion from the ferredoxin may also be obtained, for example, by denaturation of the protein.

The depositing of iron ions where desirable (e.g. fertilization of soil) may be performed simultaneously with chelation of iron ions where free iron ions are undesirable (e.g. water purification). For example, water in pipes (particularly pipes comprising iron) may contain a high level of iron ions, which may facilitate deleterious bacterial growth in the pipes. Chelation of the iron ions in the pipes by ferredoxin would substantially prevent the iron ions from being utilized by bacteria in the pipes. However, if the water in the pipes is then deposited on a soil, the ferredoxin chelating the iron ions would eventually degrade, releasing the iron in the soil, where it may have a beneficial effect.

According to an optional embodiment of the present invention, there is provided a method of chelating free iron ions in a subject in need thereof, the method comprising administering an effective amount of any of the preparations described hereinabove to the subject. Preferably, the subject in need has an iron overload disorder.

As used herein, the phrase “iron overload disorder” encompasses any disorder wherein a patient has an excessive and potentially harmful level of free iron ions in the body or in a part of the body. Exemplary iron overload disorders are siderosis, hemochromatosis, aceruloplasminemia, atransferrinemia, transfusional iron overload (e.g. iron overload caused by frequent blood transfusions, as occurs for example in thalassemia patients), iron overload associated with chronic liver disease, porphyria cutanea tarda, African iron overload and iron poisoning.

In an optional embodiment of the present invention, the ferredoxin used in the abovementioned methods for chelating iron ions is attached to a solid substrate. Methods of attaching proteins to a solid substrate are widely known in the art, and the ferredoxin nay be attached to the solid substrate by any such method. The solid substrate may be of any size, material and shape. For example, the solid substrate may be a stationary substrate. Such a solid substrate may be beneficial, for example, in that the ferredoxin attached thereto and/or the iron ions chelated thereby, would have a fixed, localized position. In an alternative example, the solid substrate may be in the form of small beads. Such a solid substrate may be beneficial, for example, in that the solid substrate has a high surface to volume ratio (and can thus be attached to a relatively large quantity of ferredoxin), and/or can enable convenient manipulation of the ferredoxin attached thereto and/or the iron ions chelated thereby (e.g. collection by centrifugation or filtration, performing chemical reactions on the ferredoxin, and/or deposition on soil).

Preparation of some embodiments of the present invention may be used in analytic applications, by virtue of their binding to iron ions.

Thus, according to another aspect of the present invention, there is provided a method of detecting an iron ion in a sample [e.g., biological sample, aqueous solution (e.g., water, wastewater, sewage), soil sample], the method comprising contacting the sample with the preparation of a preparation described hereinabove and detecting the presence of an iron ion chelated by said apo- form of said ferredoxin. Optionally, the apoferredoxin is attached to a label which is sensitive to the presence of a chelated iron ion. Suitable labels will be known to one of ordinary skill in the art. For example, a fluorescent label can be attached to the apoferredoxin such that the presence of a chelated iron ion will quench the fluorescence thereof, thereby enabling the presence of chelated iron ions to be detected by a measurement of fluorescence (see e.g., WO01/84161 and WO 04/040252 each of which is fully incorporated herein by reference).

Thus, when a fluorescent quencher is used, iron binding will change fluorescent of the solution which will be indicative of iron content. If desired a calibration curve can be generated for more accurate detection.

As used herein the term “about” refers to ±10%, except where defined otherwise.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Production of recombinant M. laminosus ferredoxin (mFd): The petF gene, which encodes ferredoxin, was isolated from M. laminosus and subcloned as a C-terminal hexahistidine fusion gene into a pET20b expression vector (Novagen), and overproduced in Escherichia coli BL21 (DE3) strain (Novagen). The transformed cells were grown in Terrific Broth (TB) containing 100 μg/ml ampicillin (selective marker) at 37° C. until the optical density (OD) at 600 nm reached the value of 0.6. 1 mM isopropyl β-D-1-thiogalactopyranoside (IPTG) was then added to induce ferredoxin expression. After 5-6 hours of induction, cells were harvested and pelleted by centrifugation at the temperature of 4° C. for 10 minutes at 6,000 rpm, using a Sorvall GSA rotor. The pellet was resuspended with lysis buffer (20 mM Tris-HCl pH 8.0, 50 mM NaCl) and broken in a French press at 2000 psi, at the temperature of 8° C. The soluble extract was clarified by centrifugation (17,000 rpm at 4° C. for 15 minutes in a Sorvall SS34 rotor) and loaded on a nickel-agarose column. The column was washed with 2 M NaCl and 20 mM Tris-HCl, pH 8.0. The protein was then eluted with a solution of 150 mM imidazole, 50 mM NaCl, and 20 mM Tris-HCl, pH 8.0.

Purification of apo-ferredoxin: The elution pool eluted from the nickel-agarose column, as described hereinabove, was loaded onto a Q-Sepharose HR 10/10 anion exchange column (Pharmacia) using Äkta Explorer. Increasing the NaCl concentration in 20 mM Tris-HCl pH 8.0 buffer, by steps to 0.45 M NaCl, induced mFd elution.

As shown in FIG. 2, two protein elution peaks were detected. The elution peak which appeared at a lower salt concentration (0.25-0.45M NaCl), also referred to herein as Peak 1, did not have any absorption at 420 nm, the characteristic wavelength for 2Fe-2S cluster absorption, whereas the later peak, also referred to herein as Peak 2, exhibited absorption at 420 nm.

The protein of each peak was re-purified on the anion exchange column. As shown in FIG. 3, spectroscopic characterization confirmed the absence of an Fe-S cluster from Peak 1, as well as the presence of an Fe-S cluster in Peak 2. The re-purified solution of Peak 1 was eluted at slightly lower ionic strength as a colorless fraction which had no detectable absorption at 420 nm (FIG. 3a). The re-purified solution of Peak 2 was red and exhibited the 420 nm absorption characteristic of an assembled 2Fe-2S cluster (FIG. 3b).

The above results indicate that Peak 1 represents apo-mFd, whereas Peak 2 represents holo-mFd.

Preliminary non-denaturing gel electrophoresis and the NMR results indicated that the apo-mFd is in a native form.

SDS-PAGE electrophoresis: In order to verify the identification of the protein of Peak 1 as apo-mFd, the proteins of Peaks 1 and 2 were examined by sodium dodecyl sulfate-polyacrylamide gel electrophoresis (SDS-PAGE) on 12.5% polyacrylamide slabs according to the Laemmli method. M. laminosus ferredoxin was used as a control.

As shown in FIG. 4, no difference was observable between the proteins of Peak 1, Peak 2 and M. laminosus ferredoxin. All three protein samples exhibited a molecular weight of approximately 14 kDa. These results confirm that the proteins of both Peak 1 and Peak 2 are M. laminosus ferredoxin, wherein Peak 1 represents the apoprotein and Peak 2 represents the holoprotein.

Apo-mFd chelation activity: 0.5 mg of apo-mFd was incubated for 24 hours with 1 liter of water comprising 1.3 ppm FeCl₂, at a pH of 6.5. At the end of the incubation period, the apo-mFd was found to have removed 99.8% of the Fe in the water. The concentration of Fe in the solution was determined by inductively coupled plasma mass spectrometry (ICP-MS).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

Aono S, Bryant F O and Adams M W W, A novel and remarkably thermostable ferredoxin from the hyperthermophilic archaebacterium Pyrococcus furiosus, J. Bacteriol. 171 (1989) pp. 3433-3439.
Busch J L H, Breton J L, Davy S L, James R, Moore G R, Armstrong F A and Thomson A J, Ferredoxin III of Desulfovibrio africanus: sequencing of the native gene and characterization of a histidine-tagged form, Biochem. J. 346 (2000) pp. 375-384.
Cammack R, Rao K K, Bargeron C P, Hutson K G, Andrew P W and Rogers L J, Midpoint redox potentials of plant and algal ferredoxins, Biochem. J. 168 (1977), pp. 205-209.
Hase T, Wakabayashi S, Matsubara H, Rao K K, Hall D O, Widmer H et al., The amino acid sequence of ferredoxin from the alga Mastigocladus laminosus, Phytochemistry 17 (1978), pp. 1863-1867.
Holden H M, Jacobson B L, Hurley J K, Tollin G, Oh B H, Skjeldal L et al., Structure-function studies of 0[2Fe-2S] ferredoxins, J. Bioenerg. Biomembr. 26 (1994), pp. 67-88.
Knaff D B and Hirasawa M, Ferredoxin-dependent chloroplast enzymes, Biochim. Biophys. Acta 1056 (1991), pp. 93-125.
Li H-M, Theg S M, Bauerle C M and Keegstra K, Metal-ion-center assembly of ferredoxin and plastocyanin in isolated chloroplasts, Proc. Natl. Acad. Sci. 87 (1990), pp. 6748-6752.
Nishio K and Nakai M, Transfer of iron-sulfur cluster from NifU to apoferredoxin, J. Biol. Chem. 275 (2000), pp. 22615-22618.
Setif P, Fischer N, Lagoutte B, Bottin H and Rochaix J D, The ferredoxin docking site of photosystem I, Biochim. Biophys. Acta 1555 (2002), pp. 204-209.

1. A pure preparation of an apo- form of a ferredoxin, said preparation exhibiting thermal stability at about 70° C.

2. The preparation of claim 1, wherein said apo- form of ferredoxin comprises a native apoferredoxin.

3. The preparation of claim 1, wherein said ferredoxin comprises an [Fe₂S₂] ferredoxin.

4. The preparation of claim 3, wherein said [Fe₂S₂] ferredoxin is derived from Mastigocladus laminosus.

5. A process for obtaining a pure preparation of an apo- form of an [Fe₂S₂] ferredoxin, the process comprising:
applying a solution comprising a holo- form of said [Fe₂S₂] ferredoxin on an anion exchange column; and
eluting said [Fe₂S₂] ferredoxin from said anion exchange column with a solution comprising a salt, thereby obtaining the pure preparation of the apo- form of the [Fe₂S₂] ferredoxin.

6. The process of claim 5, wherein said salt comprises a chloride salt.

7. The process of claim 6 wherein said chloride salt comprises NaCl.

8. The process of claim 6, wherein said solution comprises a concentration of a chloride salt that ranges from about 0.25 to about 1 M.

9. The process of claim 8, wherein said solution comprises a concentration of a chloride salt that ranges from about 0.25 to about 0.4 M.

10. The process of claim 5, further comprising collecting a fraction which comprises the apo- form of the [Fe₂S₂] ferredoxin following said eluting.

11. The process of claim 5, wherein said apo- form of an [Fe₂S₂] ferredoxin exhibits thermal stability at about 70° C.

12. The process of claim 5, wherein the holo- form of said [Fe₂S₂] ferredoxin is a recombinant protein expressed in bacteria.

13. A pure preparation of an apo- form of an [Fe₂S₂] ferredoxin produced according to the process of claim 5.

14. A method of chelating iron ions from a solution, the method comprising contacting the solution with the preparation of claim 1, thereby chelating the iron ions from the solution.

15. The method of claim 14, wherein said solution is an aqueous solution.

16. A method of fertilizing a soil, the method comprising:
(a) chelating an iron ion with the preparation of claim 1; and
(b) depositing said iron ion chelated by said apo-from of said ferredoxin on said soil;
thereby fertilizing said soil.

17. A method of chelating free iron ions in a subject in need thereof, the method comprising administering an effective amount of the preparation of claim 1 to said subject, thereby chelating free iron ions in the subject.

18. The method of claim 14, wherein said ferredoxin is attached to a solid substrate.

19. The method of claim 17, wherein the subject has an iron overload disorder.

20. The method of claim 19, wherein said iron overload disorder is selected from the group consisting of siderosis, hemochromatosis, aceruloplasminemia, atransferrinemia, transfusional iron overload, iron overload associated with chronic liver disease, porphyria cutanea tarda, African iron overload and iron poisoning.

21. A method of detecting an iron ion in a sample, the method comprising:
contacting the sample with the preparation of claim 1; and
detecting the presence of an iron ion chelated by said apo- form of said ferredoxin,
thereby detecting an iron ion in the sample.

22. The method of claim 21, wherein said apo- form of said ferredoxin is attached to a label sensitive to the presence of said iron ion chelated by said apo- form of said ferredoxin.

23. A method of chelating iron ions from a solution, the method comprising contacting the solution with the preparation of claim 13, thereby chelating the iron ions from the solution.

24. A method of fertilizing a soil, the method comprising:
(a) chelating an iron ion with the preparation of claim 13; and
(b) depositing said iron ion chelated by said apo-from of said ferredoxin on said soil;
thereby fertilizing said soil.

25. A method of chelating free iron ions in a subject in need thereof, the method comprising administering an effective amount of the preparation of claim 13 to said subject, thereby chelating free iron ions in the subject.

26. The method of claim 23, wherein said ferredoxin is attached to a solid substrate.

27. The method of claim 24, wherein said ferredoxin is attached to a solid substrate.

28. The method of claim 25, wherein said ferredoxin is attached to a solid substrate.

29. The method of claim 25, wherein the subject has an iron overload disorder.

30. A method of detecting an iron ion in a sample, the method comprising:
contacting the sample with the preparation of claim 13; and
detecting the presence of an iron ion chelated by said apo- form of said ferredoxin,
thereby detecting an iron ion in the sample.

31. The method of claim 30, wherein said apo- form of said ferredoxin is attached to a label sensitive to the presence of said iron ion chelated by said apo- form of said ferredoxin.
